(19) United States
(12) Patent Application Publication
(10) Pub. No.: US 2024/0199427 A1
(43) Pub. Date: Jun. 20, 2024

(54) SYSTEMS, METHODS, AND DEVICE FOR PYROLYSIS OF METHANE IN A MICROWAVE PLASMA FOR HYDROGEN AND STRUCTURED CARBON POWDER PRODUCTION

(71) Applicant: 6K Inc., North Andover, MA (US)

(72) Inventor: Jared Majcher, Portsmouth, NH (US)

(21) Appl. No.: 18/494,522

(22) Filed: Oct. 25, 2023

Related U.S. Application Data
(60) Provisional application No. 63/387,669, filed on Dec. 15, 2022.

(51) Int. Cl. C01B 32/184; C01B 3/24; C07C 2/78; H05H 1/30

(52) U.S. Cl. CPC C01B 32/184; C01B 3/24; C07C 2/78; H05H 1/30; C01B 2203/0272; C01B 2203/0855; C01B 2203/0861; C01B 2203/1241; C01P 2002/72; C01P 2004/03; H05H 2245/10

(57) ABSTRACT

The embodiments disclosed herein are directed to systems, methods, and devices for pyrolysis of methane in a microwave plasma for hydrogen production and structured carbon powder. Some methods are directed to producing a structured carbon powder using a microwave generated plasma comprising injecting a plasma gas comprising methane (CH₄) into a liner, the liner in communication with a microwave waveguide; propagating microwaves through the microwave waveguide, the microwaves generated using a microwave generator; and generating a microwave plasma by contacting the plasma gas with the microwaves.

### INCORPORATION BY REFERENCE TO ANY PRIORITY APPLICATIONS

This application claims the priority benefit under 35 U.S.C. § 119(e) of U.S. Provisional Application No. 63/387,669, filed Dec. 15, 2022, the entire disclosure of which is incorporated herein by reference in its entirety. Any and all applications for which a foreign or domestic priority claim is identified in the Application Data Sheet as filed with the present application are hereby incorporated by reference under 37 CFR 1.57.

### BACKGROUND

### Field

The present disclosure is generally directed to structured carbon and hydrogen and methods of production thereof.

### Description

Using methane in a plasma torch is established as a possible method for various industrial processes including hydrogen generation, acetylene formation, and many other hydrocarbon formations and carbon nanotube (CNT) formation has been shown to occur during the pyrolysis of methane under certain conditions. This would be hugely beneficial as the CNTs produced via this method would not require additional purification steps which often reduce the quality.

Microwave plasma technology has provided stable hydrogen-based plasma for many years. Additional hot zone technologies are uniquely positioned at this time to attempt the controlled pyrolysis of methane to produce bulk hydrogen and CNTs. Novel methods for producing carbon structures and hydrogen are presented herein.

### SUMMARY

For purposes of this summary, certain aspects, advantages, and novel features of the invention are described herein. It is to be understood that not all such advantages necessarily may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

Some embodiments herein are directed to methods for producing a structured carbon powder using a microwave generated plasma, the method comprising: injecting a plasma gas comprising methane (CH₄) into a liner, the liner in communication with a microwave waveguide; propagating microwaves through the microwave waveguide, the microwaves generated using a microwave generator; and generating a microwave plasma by contacting the plasma gas with the microwaves, wherein contacting the plasma gas with the microwaves pyrolyzes the plasma gas into pyrolysis products, wherein the pyrolysis products comprise hydrogen gas and solid carbon, and wherein the solid carbon comprises a sheet or platelet microstructure.

In some embodiments, a pressure within the liner and the reaction chamber is 760 Torr. In some embodiments, a frequency of the microwaves is between about 300 MHz and about 300 GHz. In some embodiments, a frequency of the microwaves is about 915 MHz.

In some embodiments, the exhausting the ionized plasma gas from the liner into the reaction chamber comprises directing the ionized plasma gas through an extension tube at an exit of the liner. In some embodiments, the extension tube comprises graphite. In some embodiments, the liner comprises quartz.

In some embodiments, the plasma gas comprises between about 0 vol % and about 5 vol %. In some embodiments, the solid carbon comprises a sheet microstructure, and wherein edges of the carbon sheets measure between about 100 nm and about 1000 nm. In some embodiments, the solid carbon comprises a sheet microstructure, and wherein edges of the carbon sheets measure between about 50 nm and about 150 nm.

In some embodiments, the solid carbon comprises substantially no amorphous carbon. In some embodiments, the solid carbon comprises substantially no carbon nanotubes. In some embodiments, the solid carbon comprises nanosheets of graphene.

In some embodiments, the pyrolysis products comprise acetylene (C₂H₂). In some embodiments, the pyrolysis products comprise at least one of CH₃, C₂H₅, and C₂H₃.

In some embodiments, the plasma gas is contacted with the microwaves in the absence of a catalyst. In some embodiments, the plasma gas comprises argon (Ar). In some embodiments, the plasma gas comprises hydrogen (H₂). In some embodiments, the plasma gas is injected into the liner at a temperature above 500° C. In some embodiments, the solid carbon is solidified in the gas phase.

### BRIEF DESCRIPTION OF THE DRAWINGS

The drawings are provided to illustrate example embodiments and are not intended to limit the scope of the disclosure. A better understanding of the systems and methods described herein will be appreciated upon reference to the following description in conjunction with the accompanying drawings, wherein:

FIG. 1 illustrates a diagram of an example swirl flow microwave plasma system according to some embodiments herein.

FIG. 2 illustrates a simplified equilibrium diagram for 1 mol of methane with solid carbon omitted according to some embodiments herein.

FIG. 3 illustrates an example diagram of the total methane decomposition reaction tree according to some embodiments herein.

FIG. 4 illustrates an embodiment of a microwave plasma torch that can be used in the production of materials according to some embodiments herein.

FIGS. 5A-B illustrate an exemplary microwave plasma torch that includes a side feeding hopper, thus allowing for downstream feeding, according to some embodiments herein.

FIG. 6 illustrates mass spectrometer results for methane level testing according to some embodiments herein.

FIG. 7 illustrates equilibrium mass spectrum data taken during Test 1b according to some embodiments herein.

FIG. 8 illustrates FESEM images taken of the carbon sample of Test 1b according to some embodiments herein.

FIG. 9 illustrates an x-ray diffraction pattern from the carbon sample taken from Test 1b according to some embodiments herein.

FIG. 10 illustrates mass spectrometry data from Test 2 through the graphite extension tube measured over 10 minutes according to some embodiments herein.

FIG. 11 illustrates FESEM images taken of the carbon sample of Test 2 according to some embodiments herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the invention is defined solely by the claims. The features illustrated or described in connection with one exemplary embodiment may be combined with the features of other embodiments. Such modifications and variations are intended to be included within the scope of the present technology.

In some embodiments, microwave plasma processes to perform pyrolysis of methane gas composition and carbon morphology are provided herein. The microwave plasma technologies described herein provide energy efficiency compared to traditional methods of material synthesis and purification, precise control over material composition. Furthermore, materials produced using the microwave plasma technology described herein may high purity levels, making them suitable for applications where material quality is critical. Furthermore, microwave plasma processes are environmentally sustainable compared to traditional processing methods due to reduced energy consumption and waste generation.

In some embodiments, the microwave plasma processes described herein may be used to produce high-quality carbon structures, such as carbon nanotubes (CNTs). Although CNTs) may be formed during carbon formation, in some embodiments, a high percentage of carbon may be converted to graphene nanoplatelets. In some embodiments, the composition of the gas products may be consistent with partial pyrolysis of methane with a high degree of acetylene production as well as hydrogen gas.

Some embodiments herein relate to the use of microwave plasma processing to produce carbon structures and bulk hydrogen. A microwave plasma at atmospheric pressure is generally considered a thermal plasma, i.e., electrons and ions have energy at the same level due to high collision frequency. However, the degree of ionization may not be exceedingly high, only about 10% of gas particles becoming ionized. While the ion energies in microwave plasmas are lower than those of higher potential direct-current (DC) arc systems, the electron energies are quite high. This makes microwave plasma good for forming radicals which can be used to enhance or even initiate certain chemical steps which would otherwise not occur at these pressures and temperatures. The chemical species present in radio frequency (RF) plasma at higher powers compared to that of lower power in microwave plasma show that a higher intensity and degree of radicals are present in the microwave plasma relative to the RF.

Plasma torches generate and provide high temperature directed flows of plasma for a variety of purposes. The two main types of plasma torches are induction plasma torches and microwave plasma torches. Generally, inductive plasmas suffer from plasma non-uniformity. This non-uniformity leads to limitations on the ability of inductive plasmas to process certain materials. Furthermore, significant differences exist between the microwave plasma apparatuses and other plasma generation torches, such as induction plasma. For example, microwave plasma is hotter on the interior of the plasma plume, while induction is hotter on the outside of the plumes. In particular, the outer region of an induction plasma can reach about 10,000 K while the inside processing region may only reach about 1,000 K. This large temperature difference leads to material processing and feeding problems. Furthermore, induction plasma apparatuses are unable to process feedstocks at low enough temperatures to avoid melting of certain feed materials without extinguishing the plasma.

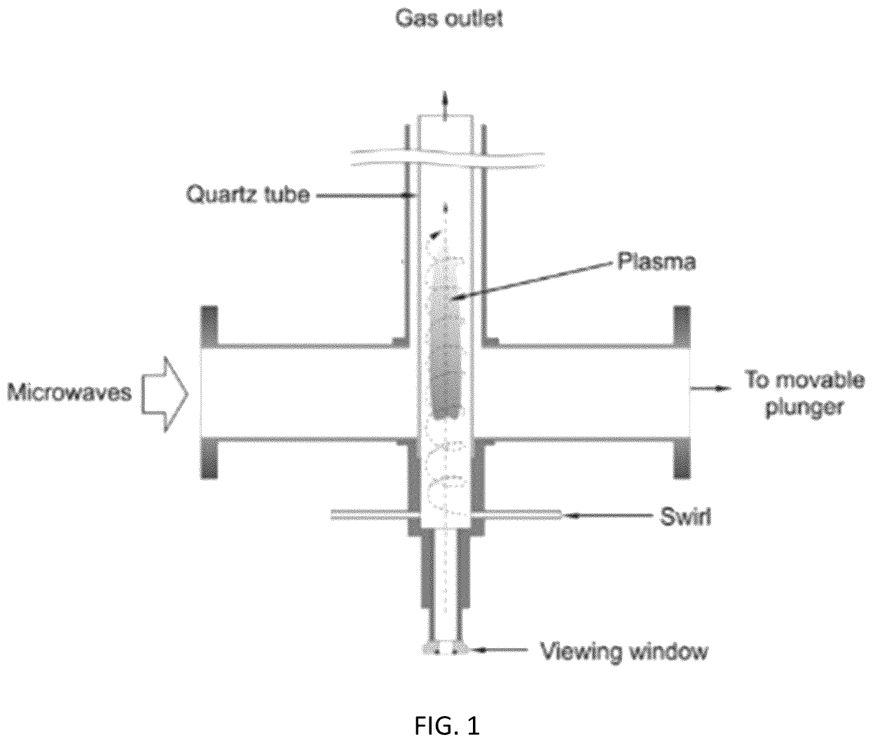

Unlike other plasma systems driven by high voltage potentials (e.g., DC arc), in some embodiments, microwave plasmas are primarily driven by the avalanche effect with an initial seed of free electrons required to begin the plasma. In some embodiments, joule heating of the gas follows as the ionized gases couple the microwave energy like an antenna and continue to heat the surrounding gases. An example microwave plasma torch setup according to some embodiment is shown in FIG. 1. In some embodiments, the plasma gas is injected into a quartz liner which runs through a microwave waveguide where microwaves are applied to the plasma gas. The ionized gas is then exhausted into a chamber where mixing and recombination of the ionized species may occur.

In some embodiments, two main methods of methane breakdown may occur: ionization and pyrolysis. Ionization of gas is the act of coupling enough energy into the gas molecule to raise an electron out of the gas molecule's orbital level. In the case of methane, there are many ionization states which can occur. For example, an electron may be stripped, creating CH₄+. Then, in some embodiments, for example, a hydrogen atom may be stripped, creating CH₃+, and so on until C+. The ionization energy required to reduce methane starts at 12.62 eV for CH₄+ and increases to 25 eV for C+. The lowest energy level is CH₄+ at 12.62 cV due to the loosely bound 112 orbital electron. In some embodiments, there may be an instability due to the Jahn-Teller effect which results in multiple products from this ionization and the eV required is between 12.51 and 12.71 eV. However, in some embodiments, since the energy level of a microwave field at, e.g., 915 MHz, is well below that level, the primary method of decomposition of methane will be through pyrolysis and not direct ionization. The energy level may be calculated according to the below equation, wherein eV is the energy in the electromagnetic filed, h is Plank's constant, c is the speed of light, and A is the wavelength of the electromagnetic waves.

$$e V = \frac{h c}{λ}$$

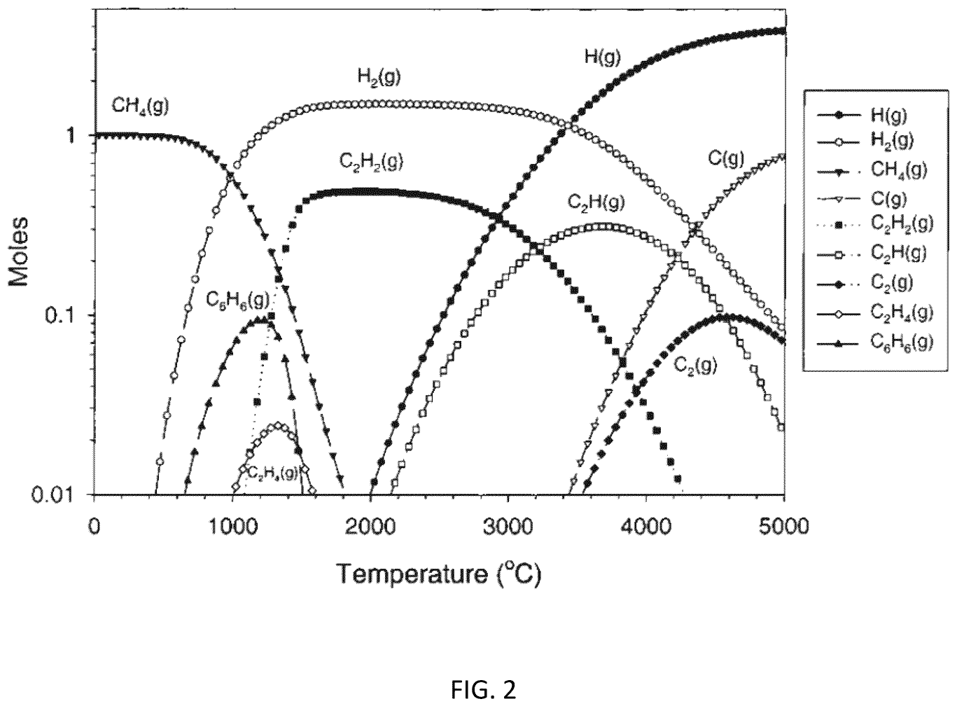

Pyrolysis of methane by simple thermal breakdown begins at 500° C. and finishes around 1000° C., and at thermodynamic equilibrium produces the by-products solid carbon and molecular hydrogen gas. The standard model referenced when discussing methane pyrolysis is by the unimolecular dissociation of hydrogen molecules from methane to gradually lower hydrogenated carbon radicals. However, this study was performed over long time periods and at temperatures much lower than those present in a microwave thermal plasma, such as that used in some embodiments herein. With short residence times, omitting solid carbon from the equilibrium diagram is more appropriate to understand the immediate concentrations leaving the plasma and is shown in FIG. 2. In some embodiments, the primary by-product besides hydrogen gas may comprise acetylene (C₂H₂) which is stable to high temperatures.

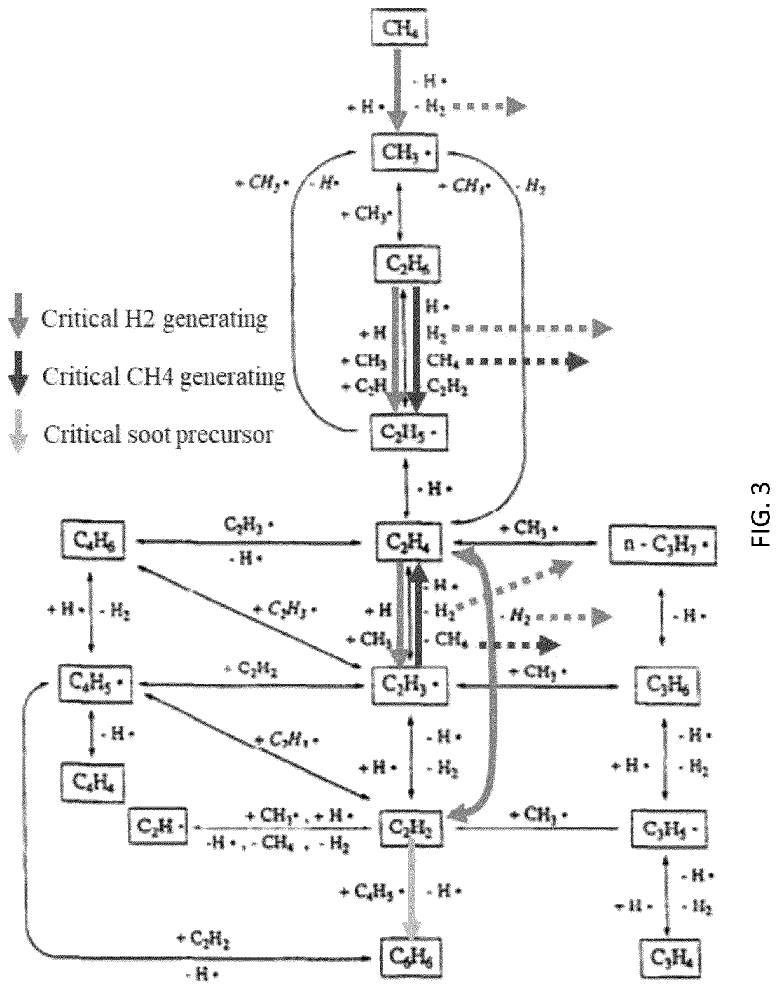

Table 1 lists the critical reactions for hydrogen generation via methane pyrolysis when considering a temperature between about 1500 K and about 2000 K over a time of about 37 ms. Modelling the pyrolysis of methane has shown that the primary source of hydrogen gas is R1 with secondary reactions with ethylene (R2) and ethane (R3, R4) decomposition accounting for about 10% or less. In some embodiments, R1 and R5 are the most important for methane conversion with over 99% of methane decomposing via these mechanisms. In some embodiments, methane decomposition is not the only process that can occur but also methane recombination. In some embodiments, reactions R6 and R7 are the primary mechanisms via which methane is shown to be generated in the complicated soup of hydrocarbons that can be present during methane decomposition. FIG. 3 illustrates an example diagram of the total methane decomposition reaction tree according to some embodiments herein.

TABLE 1
List of critical chemical reactions for methane pyrolysis.

| | | A | | E | |
|---|---|---|---|---|---|
| No. | Reaction | (mol + cm⁻3 + s¹) | n | (kcal/mol) | Critical |
| R1 | CH₄ + H ↔ CH₃ + H₂ | 2.19e−20 | 3 | 8.037 | H, M |
| R2 | C₂H₆ + H ↔ C₂H₅ + H₂ | 2.41e−15 | 0 | 7.411 | H |
| R3 | C₂H₄ + M ↔ C₂H₂ + H₂ + M | 1.65e−07 | 0 | 71.54 | H, A |
| R4 | C₂H₄ + H ↔ C₂H₃ + H₂ | 9.0e−10 | 0 | 14.9 | H |
| R5 | H + CH₃ + M ↔ CH₄ + M | 3.88e−24 | −1.8 | 0 | M |
| R6 | C₂H₆ + CH₃ ↔ C₂H₅ + CH₄ | 2.51e−31 | 6 | 6.046 | M+ |
| R7 | C₂H₄ + CH₃ ↔ CH₄ + C₂H₃ | 6.91e−12 | 0 | 11.128 | M+ |
| R8 | C₂H₂ + H + M ↔ C₂H₃ + M | 9.45e−30 | 0 | 1.47 | A |

Legend: H = hydrogen generation, M = methane conversion, A = acetylene formation, M+ = methane recombination. Arrhenius rate equation constants k(T) = ATⁿe^{E/RT}

In some embodiments, during the pyrolysis of methane, the two equilibrium products are hydrogen gas and solid carbon. In some embodiments, the form of the solid carbon is dependent on many factors. Typically, in some embodiments, if the solid carbon is quenched on a surface, coal coke is formed. In some embodiments, if the carbon is solidified in the gas phase, the formation of carbon black may occur. The most recognized method for carbon black formation is by polycyclic aromatic hydrocarbons (PAH) acting as nucleation points for soot growth. Benzene or phenyl rings lose a hydrogen atom to the monoatomic hydrogen in the gas stream and an acetylene molecule bonds to the radical site which results in surface growth of soot particles. This mechanism is referred to as the Hydrogen Abstraction Acetylene Addition (HACA). Heterogeneous nucleation of solid carbon in an acetylene flame is known to occur on PAHs, the precursor to which is benzene (C₆H₆). Solid carbon growth in this environment is assumed to progress by the adsorption of acetylene on the surface and the abstraction of hydrogen. Modelling using this assumption has shown that solid carbon growth stops once the temperature is below about 1500 K. Therefore, the presence for both benzine and acetylene may be important factors in the nucleation and growth rate of solid carbon particles. Another allotrope of carbon is CNTs whose nucleation and growth mechanisms have been studied extensively and is still debated. In the vapor-liquid-solid (VLS) model, carbon atoms are adsorbed to catalyst particles, typically a transition metal such as Ni, Fe, or Co, until supersaturated. At this point the carbon atoms may form a graphene like lattice, often called the “cap.” The cap may be pushed up and away from the catalyst particle as carbon atoms diffuse into the sp2 carbon lattice which may form. The importance of the catalyst particles is thought to have four points: preventing the closure of the carbon nanotube lattice, functionalizing the edges of the nanotube, converting the feedstock (hydrocarbon, CO, etc.) into carbon atoms, and serving as a driving force for sp2 lattice formation by supersaturating with carbon atoms.

However, in some embodiments, carbon nanotubes can be formed without a catalyst. The mechanism behind this growth is still under investigation but oxides appear to be the primary substrate. Additionally, carbon nanotubes may be created without any catalyst by heating methane through a carbon felt in a microwave chamber. Without being limited by theory, the rapid heating and the microwave environment assisted in exciting free radical species from the methane which could become nucleation points for nanotube growth. Some embodiments herein are directed to using, for example, atmospheric microwave plasma system to dissociate methane and create carbon microstructures in the resulting soot without using catalyst particles. In some embodiments, a graphite extension tube may be used to supply additional carbon radicals during methane decomposition.

### Microwave Plasma Apparatus

In some embodiments, a microwave plasma apparatus may be used in the production of carbon structures according to the embodiments herein. In some embodiments, the microwave plasma apparatus may comprise a microwave generator for generating microwaves at a frequency between 300 MHz and 300 GHz, such as, for example, 915 Mhz.

In some embodiments, the microwave plasma apparatus may comprise a gas mixing panel, plasma torch with quartz liner, water-cooled stainless-steel reactor, water cooled exhaust and water separation particle filter. In some embodiments monitoring instrumentation may be provided, including a machine vision camera, one or more thermocouples, one or more inspection ports, such as at the liner exit, and mass spectrometer, such as in the exhaust after the water-cooled sections.

In some embodiments, a graphite extension tube may be provided. In some embodiments, the extension tube extends downward into a reaction chamber of the microwave plasma apparatus, the extension tube confining and directing the microwave plasma to extend its length. In some embodiments, the extension tube may concentrate the energy and power provided by a microwave power source, in order to form a longer microwave plasma within the apparatus. The methods and apparatuses described herein may utilize an extension tube, which extends downward from a core plasma tube into the reaction chamber. In some embodiments, the extension tube may concentrate energy from the microwave power source into a smaller volume, extending and directing the plasma at a greater length than would be possible using a conventional microwave plasma apparatus. In some embodiments, a length of a plasma may be tuned or altered by configuring one or more of the following parameters: power, plasma gas flow, type of gas, extension tube material, level of insulation of the reactor chamber or the extension tube, level of coating of extension tube, and geometry of the extension tube (e.g., tapered/stepped).

Furthermore, in some embodiments, an agitator, vibrator, or other device may be provided to prevent sticking and/or to remove feedstock particles from surfaces of the extension tube. In some embodiments, providing an extension tube with a specific shape may facilitate prevention of material accumulation on one or more surfaces of the microwave plasma torch. For example, a conical extension tube may prevent buildup on surfaces of the extension tube.

In some embodiments, an extension tube as described herein may extend downward into the reaction chamber of a microwave plasma apparatus. In some embodiments, the extension tube may extend downward at a length of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the reaction chamber length, or any value between the aforementioned values.

Various parameters of the microwave plasma may be adjusted manually or automatically in order to achieve a desired material. These parameters may include, for example, power, plasma gas flow rates, type of plasma gas, presence of an extension tube, extension tube material, level of insulation of the reactor chamber or the extension tube, level of coating of the extension tube, geometry of the extension tube (e.g. tapered/stepped), feed material size, feed material insertion rate, feed material inlet location, feed material inlet orientation, number of feed material inlets, plasma temperature, residence time and cooling rates. The resulting material may exit the plasma into a sealed chamber where the material is quenched then collected. In some embodiments, the extension tube may comprise isomolded graphite and be held up by, for example, a stainless sheet scaffold. In some embodiments, the top edge of the extension tube may be machined to fit over the quartz liner to create a seamless extension of the plasma containment.

In some embodiments, the microwave plasma apparatus may be operated at atmospheric pressure (760 Torr). In some embodiments, before running, the chamber may be purged to below 100 ppm oxygen using argon gas before striking plasma. In some embodiments, after processing and once the chamber temperature has fallen below 50° C., compressed dry air (CDA) may be slowly added in 5 vol % increments with Argon until 100 vol % CDA fills the chamber.

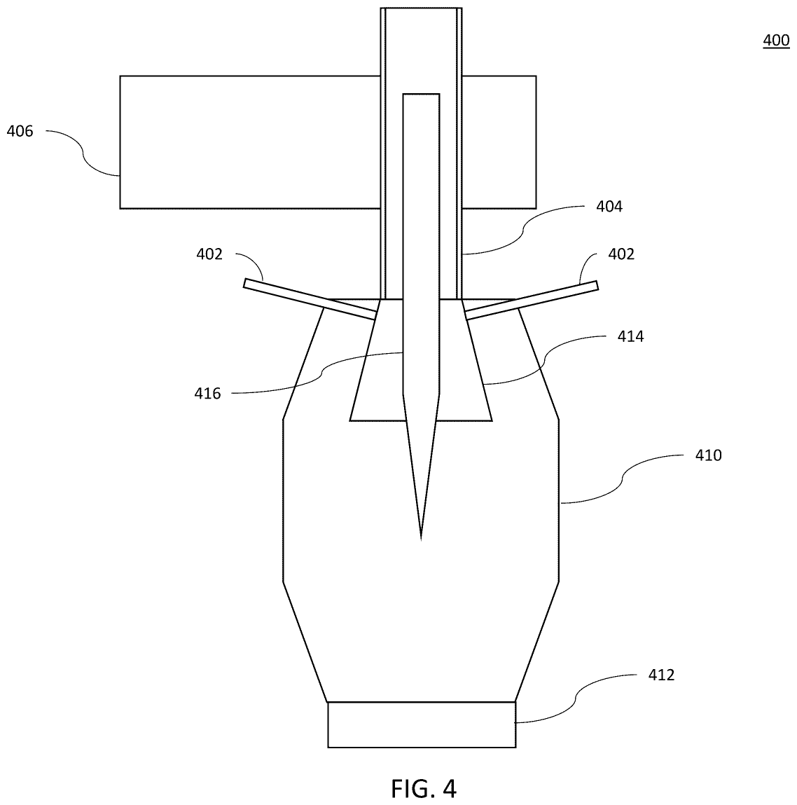

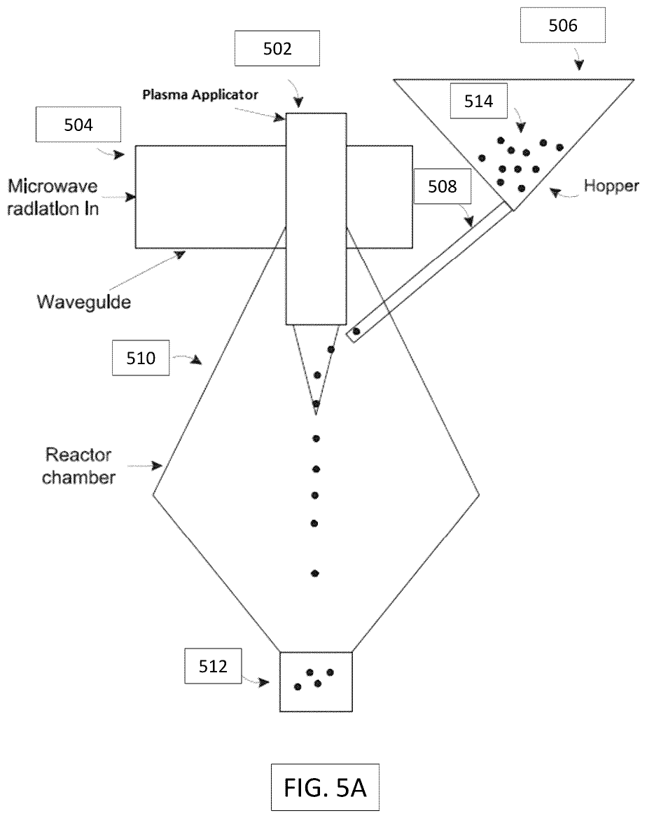

FIG. 4 illustrates an embodiment of a microwave plasma torch 400 that can be used in the production of materials according to some embodiments herein. In some embodiments, a feedstock can be introduced, via one or more feedstock inlets 402, into a microwave plasma 416. In some embodiments, an entrainment gas flow and/or a sheath flow may be injected into the microwave plasma torch 400 to create flow conditions within the plasma torch prior to ignition of the plasma 416 via microwave radiation source 406. In some embodiments, the entrainment flow and sheath flow are both axis-symmetric and laminar, while in other embodiments the gas flows are swirling. In some embodiments, the feedstock may be introduced into the microwave plasma torch 400, where the feedstock may be entrained by a gas flow that directs the materials toward the plasma 416.

The gas flows can comprise a noble gas column of the periodic table, such as helium, neon, argon, etc. Although the gases described above may be used, it is to be understood that a variety of gases can be used depending on the desired material and processing conditions. In some embodiments, within the microwave plasma 416, the feedstock may undergo a physical and/or chemical transformation. Inlets 402 can be used to introduce process gases to entrain and accelerate the feedstock towards plasma 416. In some embodiments, a second gas flow can be created to provide sheathing for the inside wall of a core gas tube or liner 404 and a reaction chamber 410 to protect those structures from melting due to heat radiation from plasma 416.

Various parameters of the microwave plasma 416 may be adjusted manually or automatically in order to achieve a desired material. These parameters may include, for example, power, plasma gas flow rates, type of plasma gas, presence of an extension tube, extension tube material, level of insulation of the reactor chamber or the extension tube, level of coating of the extension tube, geometry of the extension tube (e.g. tapered/stepped), feed material size, feed material insertion rate, feed material inlet location, feed material inlet orientation, number of feed material inlets, plasma temperature, residence time and cooling rates. The resulting material may exit the plasma into a sealed chamber 412 where the material is quenched then collected.

In some embodiments, the feedstock is injected after the microwave plasma torch applicator for processing in the “plume” or “exhaust” of the microwave plasma torch. Thus, the plasma of the microwave plasma torch is engaged at the exit end of the plasma torch core tube 404, or further downstream. In some embodiments, adjustable downstream feeding allows engaging the feedstock with the plasma plume downstream at a temperature suitable for optimal melting of feedstock through precise targeting of temperature level and residence time. Adjusting the inlet location and plasma characteristics may allow for further customization of material characteristics. Furthermore, in some embodiments, by adjusting power, gas flow rates, pressure, and equipment configuration (e.g., introducing an extension tube), the length of the plasma plume may be adjusted.

In some embodiments, feeding configurations may include one or more individual feeding nozzles surrounding the plasma plume. The feedstock may enter the plasma from any direction and can be fed in 360° around the plasma depending on the placement and orientation of the inlets 402. Furthermore, the feedstock may enter the plasma at a specific position along the length of the plasma 416 by adjusting placement of the inlets 402, where a specific temperature has been measured and a residence time estimated for providing the desirable characteristics of the resulting material.

In some embodiments, the angle of the inlets 402 relative to the plasma 416 may be adjusted, such that the feedstock can be injected at any angle relative to the plasma 416. For example, the inlets 102 may be adjusted, such that the feedstock may be injected into the plasma at an angle of about 0 degrees, about 5 degrees, about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, or about 90 degrees relative to the direction of the plasma 416, or between any of the aforementioned values.

In some embodiments, implementation of the downstream injection method may use a downstream swirl or quenching. A downstream swirl refers to an additional swirl component that can be introduced downstream from the plasma torch to keep the powder from the walls of the core tube 404, the reactor chamber 410, and/or an extension tube 414.

In some embodiments, the extension tube may extend into the reaction chamber of a microwave plasma apparatus. In some embodiments, the extension tube may comprise a stepped shape, such that the tube comprises one or more cylindrical volumes extending downward in the reaction chamber, wherein each successive cylindrical volume comprises a larger diameter than each previous cylindrical volume as the tube extends downward in the reaction chamber. In some embodiments, the extension tube may have a conical shape, tapering radially outwards as it extends downward into the reaction chamber. In some embodiments, the extension tube may comprise a single cylindrical volume.

In some embodiments, the extension tube may have a dual conical shape, where the first conical shape tapers radially outwards as it extends downward into the reaction chamber, and the second conical shape is an inverted asymmetrical shape to the first conical shape and is connected to the end of the first conical shape and tapers radially inwards as it extends downward into the reaction chamber. In some embodiments, the extension tube may comprise a dual conical shape, where the widest portion of the first conical shape is connected to the widest portion of the second conical shape. In some embodiments, the length of the first conical shape is greater than the length of the second conical shape.

In some embodiments, the extension tube may have a dual conical shape, where the first conical shape tapers radially outwards as it extends downward into the reaction chamber and the second conical shape is an inverted symmetrical shape to the first conical shape and is connected to the end of the first conical shape and tapers radially inwards as it extends downward into the reaction chamber. In some embodiments, the widest portion of the first conical shape is connected to the widest portion of the second conical shape. In some embodiments, the length of the first conical shape is equal to the length of the second conical shape. In some embodiments, the length of the second conical shape is greater than the length of the first conical shape. In some embodiments, the feed material inlets may insert feedstock within the extension tube.

In some embodiments, the extension tube may comprise a length of about 1 foot. In some embodiments, the extension tube may comprise a length of about 1 inch, about 2 inches, about 3 inches, about 4 inches, about 5 inches, about 6 inches, about 7 inches, about 8 inches, about 9 inches, about 10 inches, about 11 inches, about 1 foot, about 2 feet, about 3 feet, about 4 feet, about 5 feet, about 6 feet, about 7 feet, about 8 feet, about 9 feet, about 10 feet, about 11 feet, about 12 feet, about 13 feet, about 14 feet, about 15 feet, about 16 feet, about 17 feet, about 18 feet, about 19 feet, about 20 feet, about 21 feet, about 22 feet, about 23 feet, about 24 feet, about 25 feet, about 26 feet, about 27 feet, about 28 feet, about 29 feet, or about 30 feet, or any value between the aforementioned values.

In some embodiments, the length of a reaction chamber 410 of a microwave plasma apparatus may be about 1 foot, about 2 feet, about 3 feet, about 4 feet, about 5 feet, about 6 feet, about 7 feet, about 8 feet, about 9 feet, about 10 feet, about 11 feet, about 12 feet, about 13 feet, about 14 feet, about 15 feet, about 16 feet, about 17 feet, about 18 feet, about 19 feet, about 20 feet, about 21 feet, about 22 feet, about 23 feet, about 24 feet, about 25 feet, about 26 feet, about 27 feet, about 28 feet, about 29 feet, or about 30 feet, or any value between the aforementioned values.

In some embodiments, the length of the plasma 416, which may be extended by adjusting various processing conditions and equipment configurations, may be about 1 foot, about 2 feet, about 3 feet, about 4 feet, about 5 feet, about 6 feet, about 7 feet, about 8 feet, about 9 feet, about 10 feet, about 11 feet, about 12 feet, about 13 feet, about 14 feet, about 15 feet, about 16 feet, about 17 feet, about 18 feet, about 19 feet, about 20 feet, about 21 feet, about 22 feet, about 23 feet, about 24 feet, about 25 feet, about 26 feet, about 27 feet, about 28 feet, about 29 feet, or about 30 feet, or any value between the aforementioned values.

In some embodiments, the entrainment flow and sheath flow are both axis-symmetric and laminar, while in other embodiments the gas flows are swirling. The feed materials may be introduced axially into the microwave plasma torch, where they are entrained by a gas flow that directs the materials toward the plasma. Within the microwave generated plasma, the feed materials may be melted or partially melted in order to spheroidize the materials. Inlets can be used to introduce process gases to entrain and accelerate particles towards the plasma. In some embodiments, particles are accelerated by entrainment using a core laminar gas flow created through an annular gap within the plasma torch. A second laminar flow can be created through a second annular gap to provide laminar sheathing for the inside wall of dielectric torch to protect the wall from melting due to heat radiation from plasma. In some embodiments, the laminar flows direct particles toward the plasma along a path as close as possible to a central axis of the plasma, exposing the particles to a substantially uniform temperature within the plasma.

In some embodiments, suitable flow conditions are present to keep particles from reaching the inner wall of the plasma torch where plasma attachment could take place. In some embodiments, particles are guided by the gas flows towards the microwave plasma, where each particle undergoes homogeneous thermal treatment. Various parameters of the microwave generated plasma, as well as particle parameters, may be adjusted in order to achieve desired results. These parameters may include microwave power, feed material size, feed material insertion rate, gas flow rates, plasma temperature, residence time and cooling rates. In some embodiments, the cooling or quenching rate is not less than 10⁺³ degrees C./sec upon exiting the plasma. As discussed above, in some embodiments, the gas flows are laminar; however, in alternative embodiments, swirl flows or turbulent flows may be used to direct the feed materials toward the plasma.

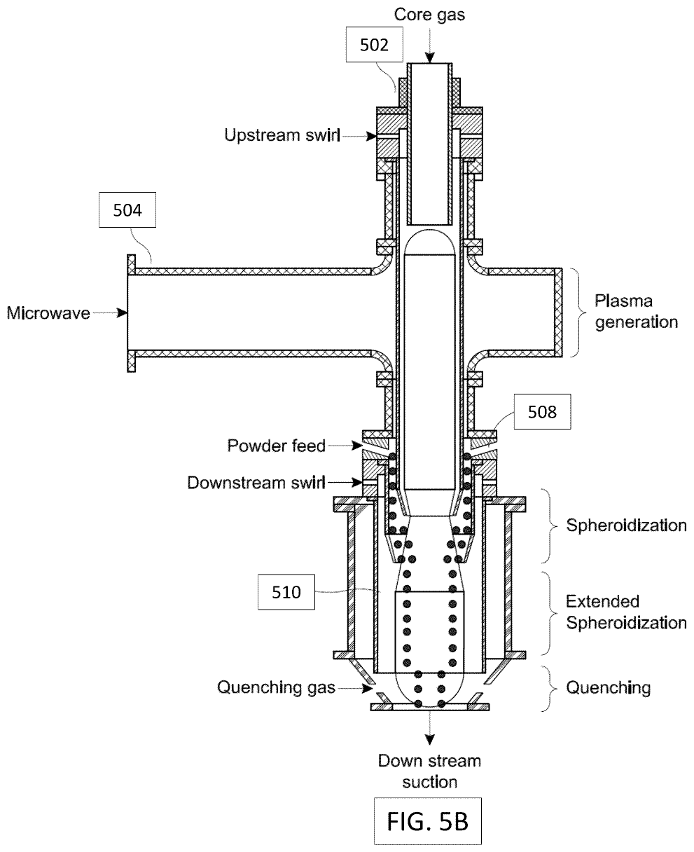

FIGS. 5A-B illustrate an exemplary microwave plasma torch that includes a side feeding hopper, thus allowing for downstream feeding. Thus, in this implementation the feedstock is injected after the microwave plasma torch applicator for processing in the “plume” or “exhaust” of the microwave plasma torch. Thus, the plasma of the microwave plasma torch is engaged at the exit end of the plasma torch to allow downstream feeding of the feedstock. This downstream feeding can advantageously extend the lifetime of the torch as the hot zone is preserved indefinitely from any material deposits on the walls of the hot zone liner. Furthermore, it allows engaging the plasma plume downstream at temperature suitable for optimal processing of powders through precise targeting of temperature level and residence time. For example, there is the ability to dial the length of the plume using microwave powder, gas flows, and pressure in the quenching vessel that contains the plasma plume.

Generally, the downstream spheroidization method can utilize two main hardware configurations to establish a stable plasma plume which are: annular torch, such as described in U.S. Pat. Pub. No. 2018/0297122, or swirl torches described in U.S. Pat. No. 8,748,785 B2 and U.S. Pat. No. 9,932,673 B2, each of which is hereby incorporated by reference in its entirety. A feed system close-coupled with the plasma plume at the exit of the plasma torch is used to feed powder axisymmetrically to preserve process homogeneity.

Other feeding configurations may include one or several individual feeding nozzles surrounding the plasma plume. The feedstock powder can enter the plasma at a point from any direction and can be fed in from any direction, 360° around the plasma, into the point within the plasma. The feedstock powder can enter the plasma at a specific position along the length of the plasma plume where a specific temperature has been measured and a residence time estimated for sufficient melting of the particles. The melted particles exit the plasma into a sealed chamber where they are quenched then collected.

The feed materials 514 can be introduced into a microwave plasma torch 502. A hopper 506 can be used to store the feed material 514 before feeding the feed material 514 into the microwave plasma torch 502, plume, or exhaust via inlet 508. The feed material 514 can be injected at any angle to the longitudinal direction of the plasma torch 502, such as at 5, 10, 15, 20, 25, 30, 35, 40, 45, 50, 55 degrees, or any value between the aforementioned values. In some embodiments, the feedstock can be injected an angle of greater than 5, 10, 15, 20, 25, 30, 35, 40, 45, 50, or 55 degrees. In some embodiments, the feedstock can be injected an angle of less than 5, 10, 15, 20, 25, 30, 35, 40, 45, 50, or 55 degrees. In alternative embodiments, the feedstock can be injected along the longitudinal axis of the plasma torch.

The microwave radiation can be brought into the plasma torch through a waveguide 504. The feed material 514 may be fed into a plasma chamber 510 and may be placed into contact with the plasma generated by the plasma torch 502. When in contact with the plasma, plasma plume, or plasma exhaust, the feed material may melt. While still in the plasma chamber 510, the feed material 514 cools and solidifies before being collected into a container 512. Alternatively, the feed material 514 can exit the plasma chamber 510 while still in a melted phase and cool and solidify outside the plasma chamber. In some embodiments, a quenching chamber may be used, which may or may not use positive pressure. While described separately from FIG. 1, the embodiments of FIGS. 5A and 5B are understood to use similar features and conditions to the embodiment of FIG. 4.

### Microwave Plasma Processing

In a microwave plasma process, the feedstock may be entrained in an inert and/or reducing gas environment and injected into the microwave plasma, the microwave plasma plume, or the microwave plasma exhaust. Upon injection into a hot plasma (or plasma plume or exhaust), the feedstock may undergo a physical and/or chemical transformation (e.g., spheroidization). After processing, the resulting material may be released into a chamber filled with an inert gas and directed into hermetically sealed drums where is it stored. This process can be carried out at atmospheric pressure, in a partial vacuum, or at a slightly higher pressure than atmospheric pressure.

In alternative embodiments, the process can be carried out in a low, medium, or high vacuum environment. The process can run in batches or continuously, with the drums being replaced as they fill up with processed material. By controlling the process parameters, such as cooling gas flow rate, residence time, plasma conditions, cooling gas composition, various material characteristics can be controlled.

Residence time of the particles within a hot zone of the plasma can also be adjusted to provide control over the resulting material characteristics. That is, the length of time the particles are exposed to the plasma determines the extent of melting of the feedstock particles (i.e., surface of the particle melted as compared to the inner most portion or core of the particle). Residence time can be adjusted by adjusting such operating variables of particle injection rate and flow rate (and conditions, such as laminar flow or turbulent flow) within the hot zone. Equipment changes can also be used to adjust residence time. For example, residence time can be adjusted by changing the cross-sectional area of the plasma, by, for example, extending the plasma. In some embodiments, extending the plasma may comprise incorporating an extension tube into the microwave plasma apparatus.

In some embodiments, the extension tube may comprise a stepped shape, such that the tube comprises one or more cylindrical volumes extending downward in the reaction chamber, wherein each successive cylindrical volume comprises a larger diameter than each previous cylindrical volume as the tube extends downward in the reaction chamber. In some embodiments, the extension tube may have a conical shape, tapering radially outwards as it extends downward into the reaction chamber. In some embodiments, the extension tube may comprise a single cylindrical volume 4. In some embodiments, the feed material inlets may insert feedstock within the extension tube.

In some embodiments, the extension tube may comprise a length of about 1 foot. In some embodiments, the extension tube may comprise a length of about 1 inch, about 2 inches, about 3 inches, about 4 inches, about 5 inches, about 6 inches, about 7 inches, about 8 inches, about 9 inches, about 10 inches, about 11 inches, about 1 foot, about 2 feet, about 3 feet, about 4 feet, about 5 feet, about 6 feet, about 7 feet, about 8 feet, about 9 feet, about 10 feet, about 11 feet, about 12 feet, about 13 feet, about 14 feet, about 15 feet, about 16 feet, about 17 feet, about 18 feet, about 19 feet, about 20 feet, about 21 feet, about 22 feet, about 23 feet, about 24 feet, about 25 feet, about 26 feet, about 27 feet, about 28 feet, about 29 feet, or about 30 feet, or any value between the aforementioned values.

In some embodiments, the feedstock particles are exposed to a temperature profile at between 4,000 and 8,000 K within the microwave plasma. In some embodiments, the particles are exposed to a temperature profile at between 3,000 and 8,000 K within the microwave plasma. In some embodiments, one or more temperature sensors may be located within the microwave plasma torch to determine a temperature profile of the plasma.

### EXAMPLES

Tests were conducted using a microwave plasma apparatus comprising a 915 MHz microwave generator, a gas mixing panel, plasma torch with quartz liner, water-cooled stainless-steel reactor, water cooled exhaust and water separation particle filter. The monitoring instrumentation included a 1.3-megapixel machine vision camera, midbody type K thermocouple, inspection port at the liner exit, and mass spectrometer in the exhaust after the water-cooled sections. 3″ID/4″ OD×6″ long tube made of E+25 isomolded graphite was used as the extension and held up by 304 stainless sheet scaffold. The top edge was machined to fit over the quartz liner to create a seamless extension of the plasma containment.

All tests were operated at atmospheric pressure (760 Torr). Before running, the chamber was purged to below 100 ppm oxygen using argon gas before striking plasma. After the tests and once the chamber temperature had fallen below 50 C, compressed dry air (CDA) was slowly added in 5 vol % increments with Argon until 100 vol % CDA filled the chamber. Table 2 lists process conditions for the various tests.

TABLE 2
Test Process Conditions

| | Power, | H₂ | Ar | CH₄ | Flow Rate | Runtime |
|---|---|---|---|---|---|---|
| Test | net kW | Vol % | Vol % | Vol % | SCFM (Ar) | (min) |
| 1a | 8 | 20 | 0 | 80 | 5 | <1 |
| 1b | 12.5 | 20 | 75-77 | 3-5 | 9 | 20 |
| 2 | 12.5 | 20 | 77 | 3 | 9 | 10 |

Tests 1a and 1b were conducted to determine the plasma stability of methane gas in a microwave plasma. An argon/hydrogen plasma was run at 10 kW of power. The plasma composition was to be shifted from 20 vol % to 100 vol % hydrogen. Once stable, 100% hydrogen plasma was established, and the methane composition was raised with the goal of 100% methane plasma.

Maximum flow conditions and minimum flow conditions were used to evaluate the effect on CNT synthesis as well as conversion efficiency of methane into C and H₂. The results of this test were used to determine the optimum operating conditions for Test 2. Additionally, soot samples were collected from the chamber cover, chamber walls, and collection cup and were evaluated for morphology of the carbon and any presence of CNTs.

Test 2 utilized the stable plasma conditions and integrated a graphite extension tube at the plasma liner exit. High and low methane decomposition conditions were to be tested and samples taken. Table 2 lists the process conditions used for the testing.

The gas composition was sampled using a MKS Cirrus 3 Mass Spectrometer operating at 40 kV and sampling from 1-50 AMU. The gas sample was taken at the end of the water-cooled exhaust to ensure all products were stable. Fragmentation patterns for hydrogen, methane, acetylene, and argon gas were taken from the NIST database and summed to determine composition of the exhaust. Soot samples were collected dry from the cover, wall, and collection cup of the chamber. The samples were inspected using a field emission scanning electron microscope (FESEM) to evaluate morphology. Carbon samples were suspended in isopropyl alcohol and sonicated for 5 minutes. The solution was deposited onto a single crystal silicon substrate and let dry at room temperature. To determine carbon form, the sample was evaluated using an x-ray diffractometer.

In Test 1a. Argon supply was switched directly to methane during the running (80 vol % CH₄, 20 vol % H₂). The power was set to 10 KW and the flows were 5 SCFM (measured as “methane”). The plasma was run for approximately 1 minute before extinguishing. Inspection revealed significant fowling of the quartz liner. Run length was insufficient to receive mass spectrometer data or carbon samples. The glowing liner indicated the liner temperature was exceeding 800° C. The soot was wiped away from the outside of the liner and the arcing pattern was facing the incident wave from the microwave generator.

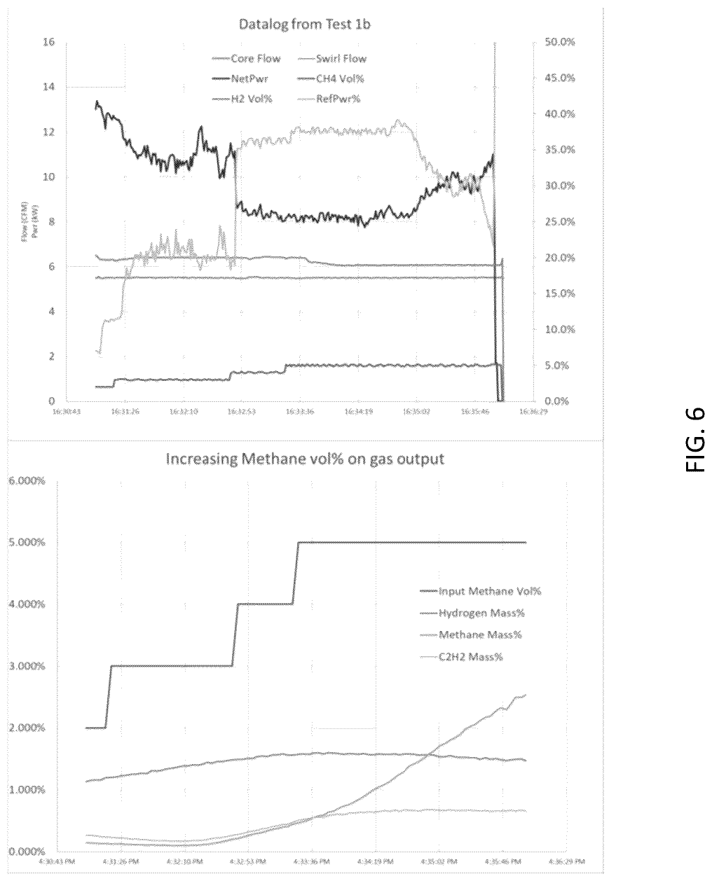

In Test 1b, methane was added to a stable Ar:H₂ plasma at low levels and increased until plasma stability suffered. 3 vol % methane in 20 vol % H₂:Ar at 13 kW was found to be the most stable condition. Increasing to 4 vol % methane resulted in high reflected power which required retuning of the microwave circuit and continuing further to 5 vol % methane resulted in abrupt failures of the plasma. FIG. 6 illustrates mass spectrometer results for the methane level testing.

As methane level was increased, total hydrogen production appears to have plateaued at a 3 vol % methane plasma and acetylene production at a 4 vol % methane plasma. The microwave circuit required tuning to bring coupled power back to 13 kW between 3 and 4 vol %. Even with tuning, plasma diameter may compress significantly with increased methane levels. Stable conditions determined during this test were then run until equilibrium in the gas products was seen from the mass spectrometer. This required approximately 10 minutes of run time.

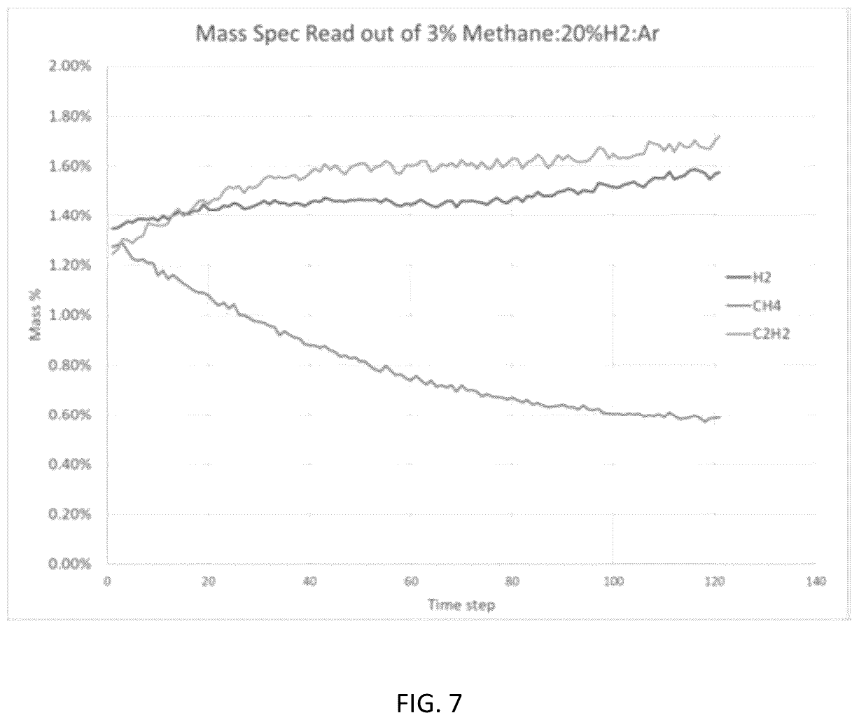

FIG. 7 illustrates equilibrium mass spectrum data taken during Test 1b. The total methane conversion was approximately 60%. Hydrogen levels increased from about 1.27 wt. % to about 1.58 wt. % and acetylene level ended at 1.72 wt. %.

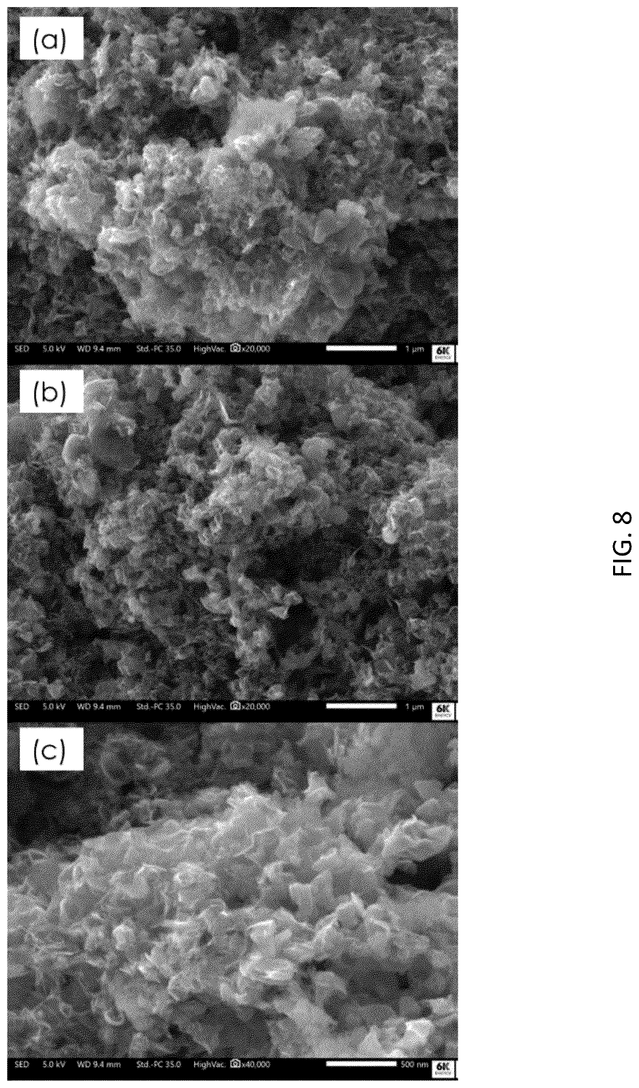

Carbon samples were taken from the cover and walls of the reactor. The total mass of sample material was less than 1 gram. Under FESEM magnification, the structure can be seen in FIG. 8 as primarily sheet or platelet like with little spherical amorphous carbon black in the field of view. No tube-like structures were observed. Exposed sheet edges measure several hundred nanometers on average. It should be noted that at higher excitation voltages above 10 kV, significant interference was observed due to material charging.

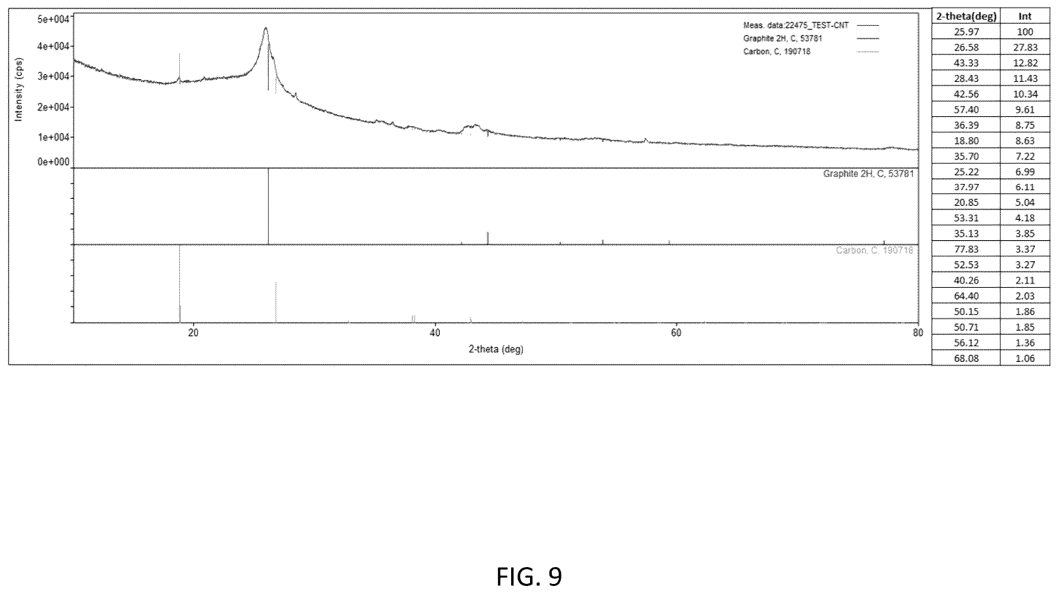

X-ray diffraction results for the carbon sample are shown in FIG. 9. The characteristic peaks of graphite and carbon are also presented to compare to the recorded signal. Although the 25.97° peak is close to the primary graphite peak, it is slightly shifted. Additionally, the primary peak for carbon at 17° is absent from the recorded signal, indicating an absence of disordered carbon in the sample.

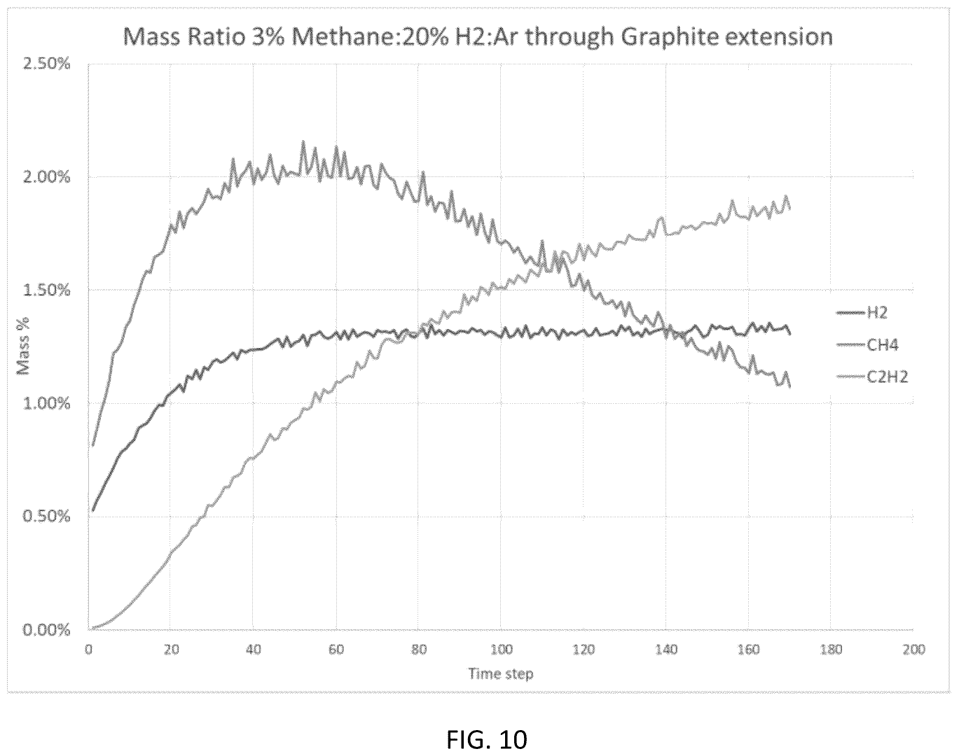

In Test 2. A graphite extension was installed and the stable plasma conditions from Test 1b were repeated. FIG. 10 illustrates mass spectrometry data from Test 2 through the graphite extension tube measured over 10 minutes. The hydrogen level increased only slightly from about 1.27 wt. % to about 1.35 wt. %. Acetylene peaked at 1.86 wt. % and methane continued to fall to 1.07 wt. % from a high of 2.16 wt. %. This signal was measured from the start of the plasma to the end of the test.

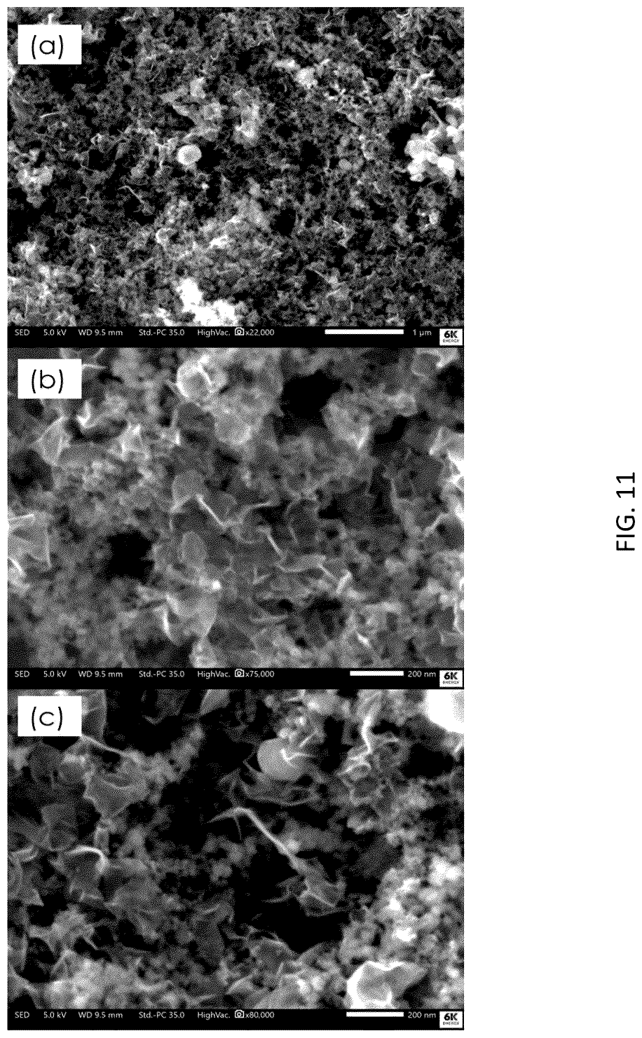

FESEM of the carbon samples collected from this test are shown in FIG. 11. Amorphous carbon/carbon black is seen in these samples as well as the sheet-like structure from test 1b. The average edge length was shorter than that seen in Test 1b and is closer to about 100 nm. No tube structures were observed.

In Test 1b, the rising hydrogen signal was a sign that some portion of the methane was converted to hydrogen and the strong acetylene signal was evidence that that the total energy or residence time of the methane was not high enough to completely pyrolyze to H₂ and carbon. Increasing the methane ratio in the plasma gases resulted in significant compression and increased reflected microwave energy. It was also noted that the plasma appeared hollow at low methane levels. It is possible that the carbon particles newly liberated from the methane gas acted to absorb or reflect the microwave energy like a conductor inserted into the microwave channel. This would explain the change in color of the plasma from a white/blue to a dull orange as the gas was no longer ionizing and instead the carbon was being heated and glowing.

While no nanotube formations were found from the testing, neither were the samples primarily amorphous carbon. Instead, the FESEM images appeared to show thin sheets which were crumpled over each other.

A critical outcome from these tests was the presence of a high concentration of nanoplatelet graphene from the pyrolysis of methane in a microwave plasma. The importance of hydrogen on the formation of gas phase nanographene most likely contributed to the creation of graphene over carbon nanotubes or carbon black.

### ADDITIONAL EMBODIMENTS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and subcombinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A method for producing a structured carbon powder using a microwave generated plasma, the method comprising:
injecting a plasma gas comprising methane (CH₄) into a liner, the liner in communication with a microwave waveguide;
propagating microwaves through the microwave waveguide, the microwaves generated using a microwave generator; and
generating a microwave plasma by contacting the plasma gas with the microwaves,
wherein contacting the plasma gas with the microwaves pyrolyzes the plasma gas into pyrolysis products,
wherein the pyrolysis products comprise hydrogen gas and solid carbon, and
wherein the solid carbon comprises a sheet or platelet microstructure.

2. The method of claim 1, wherein a pressure within the liner and the reaction chamber is 760 Torr.

3. The method of claim 1, wherein a frequency of the microwaves is between about 300 MHz and about 300 GHz.

4. The method of claim 1, wherein a frequency of the microwaves is about 915 MHz.

5. The method of claim 1, wherein the exhausting the ionized plasma gas from the liner into the reaction chamber comprises directing the ionized plasma gas through an extension tube at an exit of the liner.

6. The method of claim 5, wherein the extension tube comprises graphite.

7. The method of claim 1, wherein the liner comprises quartz.

8. The method of claim 1, wherein the plasma gas comprises between about 0 vol % and about 5 vol %.

9. The method of claim 1, wherein the solid carbon comprises a sheet microstructure, and wherein edges of the carbon sheets measure between about 100 nm and about 1000 nm.

10. The method of claim 1, wherein the solid carbon comprises a sheet microstructure, and wherein edges of the carbon sheets measure between about 50 nm and about 150 nm.

11. The method of claim 1, wherein the solid carbon comprises substantially no amorphous carbon.

12. The method of claim 1, wherein the solid carbon comprises substantially no carbon nanotubes.

13. The method of claim 1, wherein the solid carbon comprises nanosheets of graphene.

14. The method of claim 1, wherein the pyrolysis products comprise acetylene (C₂H₂).

15. The method of claim 1, wherein the pyrolysis products comprise at least one of CH₃, C₂H₅, and C₂H₃.

16. The method of claim 1, wherein the plasma gas is contacted with the microwaves in the absence of a catalyst.

17. The method of claim 1, wherein the plasma gas comprises argon (Ar).

18. The method of claim 1, wherein the plasma gas comprises hydrogen (H₂).

19. The method of claim 1, wherein the plasma gas is injected into the liner at a temperature above 500° C.

20. The method of claim 1, wherein the solid carbon is solidified in the gas phase.
